(19) United States
(12) Patent Application Publication
(10) Pub. No.: US 2009/0221875 A1
(43) Pub. Date: Sep. 3, 2009

(54) ENDOSCOPE LIGHT SOURCE SYSTEM AND ENDOSCOPE UNIT

(75) Inventor: Shotaro KOBAYASHI, Tokyo (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/392,205

(22) Filed: Feb. 25, 2009

(30) Foreign Application Priority Data
Feb. 29, 2008 (JP) 2008-050210

(51) Int. Cl. A61B 1/06

(52) U.S. Cl. 600/180

(57) ABSTRACT

An endoscope light source system including first and second light sources and an adjustment circuit is provided. The first and second light sources make first and second lights incident on an incident end of a light guide mounted in an endoscope, respectively. The first and second lights have first and second wavelength bands, respectively. The adjustment circuit adjusts the incident amounts of the first and/or second lights to satisfy a first relation if the first and second lights are simultaneously made on the incident end. The incident amounts of the first and second lights are the amounts of the first and second lights made incident on the incident end.

### BACKGROUND OF THE INVENTION

1. Field of the Invention

The present invention relates to an endoscope light source system which supplies white light and excitation light to a single light guide with a simple configuration, and which enables both a normal endoscope and an autofluorescence endoscope to produce an acceptable white light image.

2. Description of the Related Art

There is known an autofluorescence endoscope which enables a user to observe an optical autofluorescence image from tissue by shining excitation light, such as ultraviolet light, onto the tissue. In order to transmit excitation light for illuminating a subject around a peripheral area of an insert tube, a light guide is mounted in an autofluorescence endoscope. The light guide is used for transmitting white light for illuminating a subject in order to generate a normal image.

A mirror is mounted which can be inserted into and removed from the optical path of the white light in order to make either white light or excitation light incident on the light guide. When the mirror is removed from the optical path, white light strikes the light guide. On the other hand, when the mirror is inserted into the optical path, the excitation light is reflected and made incident on the light guide. However, since a mechanism for moving the mirror is necessary, the structure of the light source apparatus increases in size and complexity.

Japanese Unexamined Patent Publications Nos. 2005-342033 and 2005-342034 propose that a dichroic mirror which reflects only the excitation light component is fixed on an optical path. In such a structure, if an autofluorescence image is desired, only excitation light reflected by the dichroic mirror is made incident on the light guide. On the other hand, if a normal image is desired, white light passing through the dichroic mirror is made incident on the light guide and the excitation light is turned off. Since a mechanism for moving a mirror is unnecessary, the structure of the light source apparatus can be smaller and simpler.

However, the problem arises that an acceptable white light image cannot be produced because the components of the white light in the spectral range of the excitation light are reflected by the dichroic mirror.

### SUMMARY OF THE INVENTION

Therefore, an object of the present invention is to provide an endoscope light source system that supplies white light and excitation light to a light guide, has a simple configuration, and enables both a normal endoscope and an autofluorescence endoscope to produce an acceptable white light image.

According to the present invention, an endoscope light source system comprising first and second light sources and an adjustment circuit, is provided. The first and second light sources respectively make first and second lights incident on an incident end of a light guide mounted in an endoscope. The first and second lights have first and second wavelength bands, respectively. The adjustment circuit adjusts the incident amounts of the first and/or second lights to satisfy a first relation if the first and second lights are simultaneously made on the incident end. The incident amounts of the first and second lights are the amounts of the first and second lights made incident on the incident end.

Furthermore, the first relation is determined so that the emission amounts of the first and second lights satisfy a second relation upon making the first and second lights simultaneously incident on the incident end. The emission amounts of the first and second lights are the amounts of the first and second lights emitted from an exit end of the light guide, respectively.

A ratio of the emission amount of the first light to the emission amount of the second light is constant in the second relation.

### BRIEF DESCRIPTION OF THE DRAWINGS

The objects and advantages of the present invention will be better understood from the following description, with reference to the accompanying drawings in which:

FIG. 1 is a perspective view of an endoscope unit having an endoscope light source system of an embodiment of the present invention;

FIG. 2 is a block diagram showing the internal structure of a light-source unit;

FIG. 3 is a spectrograph showing the reflectance of the dichroic mirror;

FIG. 4 is a spectrograph showing the spectroscopic properties of the excitation light;

FIG. 5 is a spectrograph showing the spectroscopic properties of light emitted by the light-source unit when the white light and the excitation light are simultaneously emitted by the lamp and the laser source;

FIG. 6 is a graph showing the relationship between the aperture ratio and the amount of white light passing through the diaphragm;

FIG. 7 is a graph showing the relationship between the duty of the laser source and the amount of the excitation light emitted by the laser source;

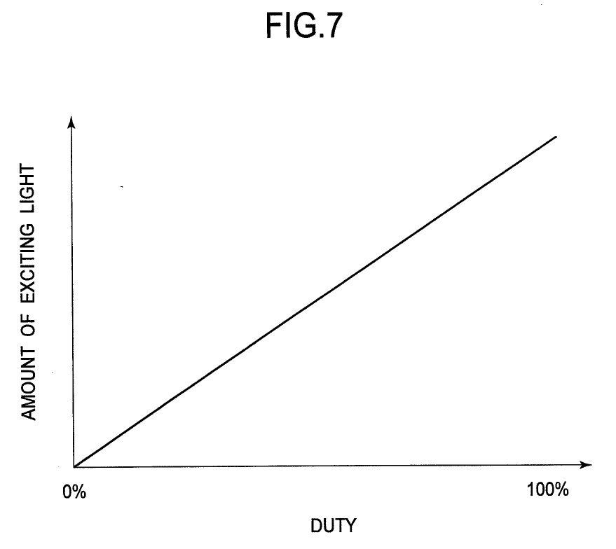

FIG. 8 is a first flowchart illustrating the initialization operation for white balance carried out by the system controller;

FIG. 9 is a second flowchart illustrating the initialization operation for white balance carried out by the system controller; and

FIG. 10 is a flowchart illustrating the operation for control of light amount carried out by the system controller when a captured image is displayed using a normal endoscope;

### DESCRIPTION OF THE PREFERRED EMBODIMENTS

The present invention is described below with reference to the embodiment shown in the drawings.

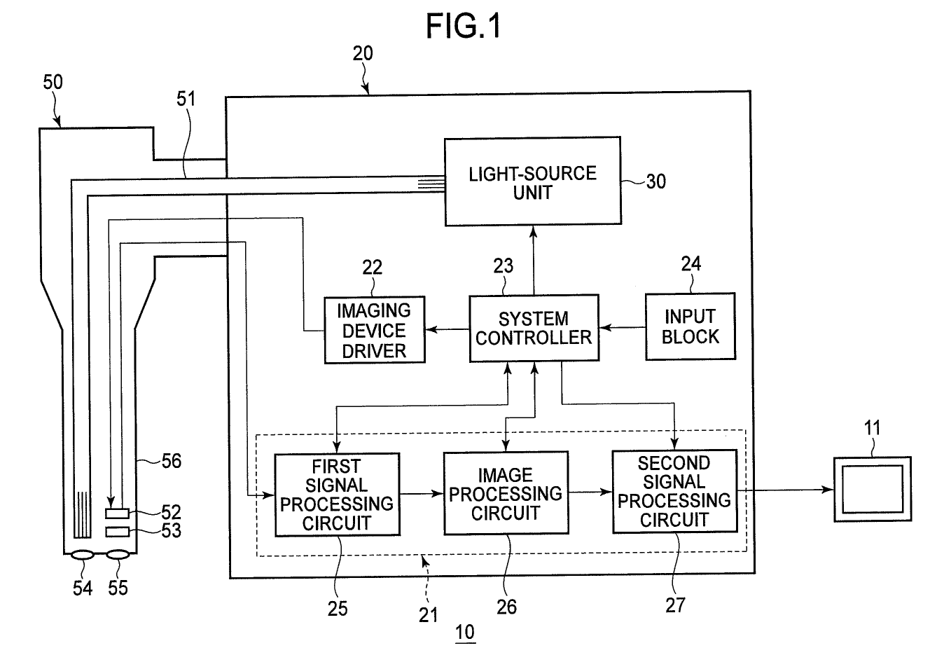

In FIG. 1, an endoscope unit 10 comprises an endoscope processor 20, an electronic endoscope 50, and a monitor 11. The endoscope processor 20 is connected to the electronic endoscope 30 and the monitor 11.

The endoscope processor 20 emits light to illuminate a desired subject. An optical image of the illuminated subject is captured by the electronic endoscope 50, and then the electronic endoscope 50 generates an image signal. The image signal is sent to the endoscope processor 20.

The endoscope processor 20 carries out predetermined signal processing on the received image signal. The image signal, having undergone predetermined signal processing, is sent to the monitor 11, where an image corresponding to the received image signal is displayed.

The endoscope processor 20 comprises a light-source unit 30, an image-processing unit 21, an imaging device driver 22, a system controller 23 (the determination circuit), an input block 24 (the switch), and other components.

As described below, the light-source unit 30 emits light for illuminating a subject toward an incident end of a light guide 51. In addition, as described below, the image-processing unit 21 carries out predetermined signal processing on the image signal. The imaging device driver 22 drives an imaging device 52 (detector) to capture an optical image of the subject. The system controller 21 controls the operations of all components of the endoscope unit 10. Following the user's input to the input block 24, various functions of the endoscope unit 10 are carried out.

The light-source unit 30 and a light-guide 51 mounted in the electronic endoscope 30 are optically connected by connection of the endoscope processor 20 to the electronic endoscope 50. This connection also results in, electrical connections being made between the image-processing unit 21 and the imaging device 52 mounted in the electronic endoscope 50, and between the imaging device driver 22 and the imaging device 52.

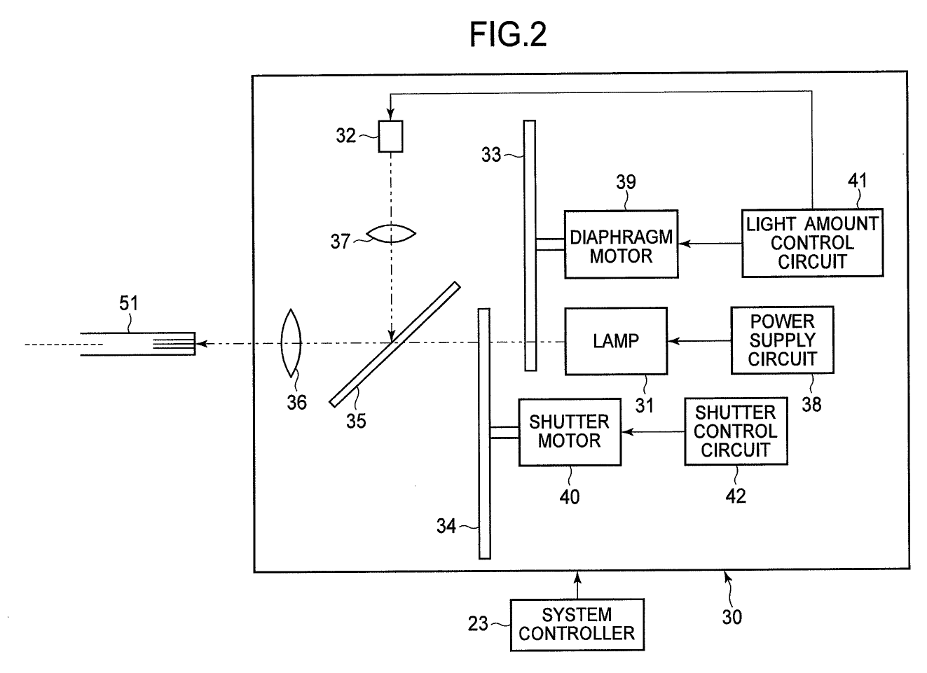

As shown in FIG. 2, the light-source unit 30 comprises a lamp 31 (the first light source), a laser source 32 (the second light source), a diaphragm 33, a rotary shutter 34, a dichroic mirror 35, a condenser lens 36, a collimator lens 37, a power supply circuit 38, a diaphragm motor 39, a shutter motor 40, a light-amount control circuit 41 (the adjustment circuit), a shutter control circuit 42, and other components.

The lamp 31, such as a xenon lamp or a halogen lamp, emits white light (the first light). The diaphragm 33, the rotary shutter 34, a dichroic mirror 35, and the condenser lens 36 are mounted on the optical path between the lamp 31 and the incident end of the light guide 51.

The amount of the white light incident on the incident end is controlled by adjusting the aperture ratio of the diaphragm 33. The aperture ratio of the diaphragm 33 is adjusted by the motor 39. The movement of the motor 39 for driving the diaphragm 33 is controlled by the light-amount control circuit 41.

As described later, the amount of light received by the imaging device 52 is communicated to the light-amount control circuit 41 via the system controller 23. The light-amount control circuit 41 controls the aperture ratio on the basis of the communicated amount of light.

The rotary shutter 34 has an aperture area and a blocking area. When white light should be allowed to pass, the aperture area is inserted into the optical path of the white light. When white light should be blocked, the blocking area is inserted into the optical path of the white light.

The rotary shutter 34 is rotated by the shutter motor 40. The passage and blocking of the white light from the light-source unit 30 are alternated by driving the shutter motor 40. The movement of the shutter motor 40 is controlled by the shutter control circuit 42. The shutter control circuit 42 is controlled by the system controller 23.

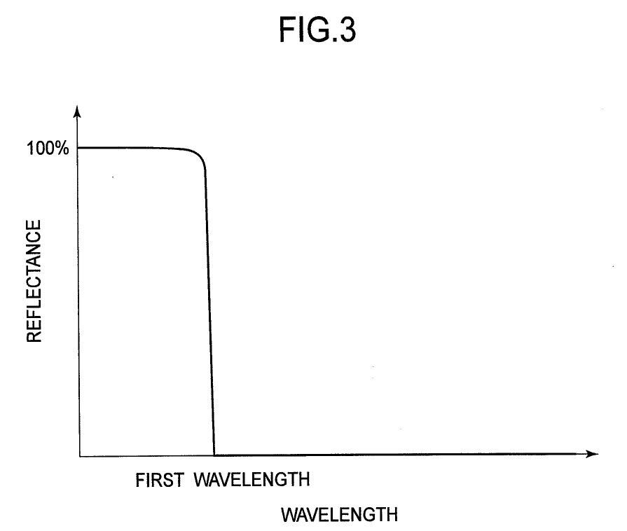

The dichroic mirror 35 is fixed so that the angle between the surface of the dichroic mirror 35 and the optical path of the white light is 45 degrees. As shown in FIG. 3., the dichroic mirror 35 reflects light of a wavelength band less than or equal to a first wavelength, and passes light of a wavelength band greater than the first wavelength. Accordingly, a first light component, which is included in the white light emitted from the lamp 31 and whose wavelength band is greater than the first wavelength, passes through the dichroic mirror 35. A second light component, which is included in the white light emitted from the lamp 31 and whose wavelength band ranges less than and equal to the first wavelength, is reflected by the dichroic mirror 35.

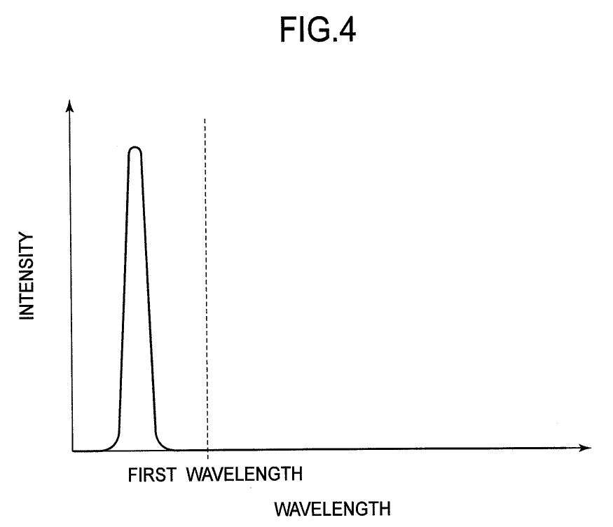

The laser source 32 emits excitation light (second light) which makes tissue autofluoresce. The excitation light is blue, and the wavelength band of the excitation light ranges below the first wavelength, as shown in FIG. 4. Accordingly, the dichroic mirror 35 reflects the excitation light. The laser source 32 is fixed so that the excitation light reflected by the dichroic mirror 35 strikes the incident end of the light guide 51.

The collimator lens 37 is mounted in the optical path between the laser source 32 and the dichroic mirror 35. The collimator lens 37 collimates the excitation light emitted by the laser source 32.

The white light component passing through the dichroic mirror 35 and/or the excitation light reflected by the dichroic mirror 35 is condensed by the condenser lens 36, and is directed to the incident end of the light guide 31.

The power supply circuit 38 supplies the lamp 31 with power. The system controller 23 controls the supply of power, and switches the lamp 31 on and off.

The laser source 32 is driven by the light-amount control circuit 41. The amount of excitation light emitted by the laser source 32 is controlled by the light-amount control circuit 41. As described later, the duty of the laser source 32 is adjusted according to the aperture ratio of the diaphragm 33, and the amount of the emitted excitation light is controlled. As described later, a corresponding relation between the duty and the aperture ratio is determined on an initialization operation for white balance.

When a normal endoscope is connected to the endoscope processor 20, only a white-light image can be observed. When the autofluorescence endoscope is connected to the endoscope processor 20, either a white-light image or an autofluorescence image can be observed. In addition, a white-light image and an autofluorescence image may be simultaneously displayed, or a false color image generated by synthesizing a white-light image and an autofluorescence image may be displayed.

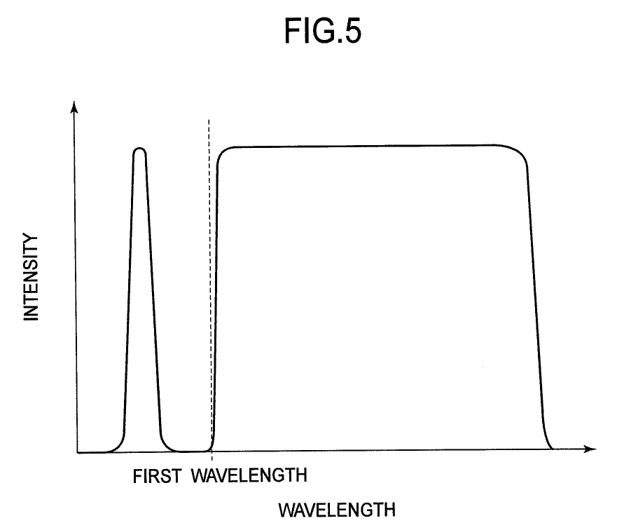

When a white-light image is to be observed, the shutter control circuit 42 orders the rotary shutter 34 to pass the white light by inserting the aperture area into the optical path, and the light-amount control circuit 41 orders the laser source 32 to emit the excitation light. As a result, the first light component and the excitation light arrive at the incident end of the light guide 51 (see FIG. 5).

On the other hand, when an autofluorescence image is to be observed, the shutter control circuit 42 orders the rotary shutter 34 to block the white light by inserting the blocking area into the optical path, and the light-amount control circuit 41 orders the laser source 32 to emit the excitation light. As a result, the excitation light is made incident on the incident end of the light guide 51 (see FIG. 4).

Next, the structure of the electronic endoscope 50, an autofluorescence endoscope, is explained in detail. As shown in FIG. 1, the electronic endoscope 50 comprises the light guide 51, the imaging device 52, an exciting-light cut-off filter 53, a diffuser lens 54, an object lens 55, and other components.

The incident end of the light guide 51 is mounted in a connector (not depicted) which connects the electronic endoscope 50 to the endoscope processor 20. And the other end, hereinafter referred to as the exit end, is mounted at the head end of the insertion tube 56 of the electronic endoscope 50. As described above, the first light component and/or the excitation light emitted by the light-source unit 30 arrives at the incident end of the light guide 51. The light is then transmitted to the exit end. The light transmitted to the exit end illuminates a peripheral area near the head end of the insertion tube 56 through a diffuser lens 54.

The light reflected by the subject illuminated by the first light component and/or the autofluorescence of the subject illuminated by the excitation light reaches the light-receiving surface of the imaging device 53 through the object lens 36 and the exciting-light cut-off filter 34, and forms an optical image on the light-receiving surface.

The imaging device driver 22 is controlled by the system controller 23, and transmits a driving signal to the imaging device 52. The imaging device 52 captures an optical image on the light-receiving surface on the basis of the received driving signal, and generates an image signal. The generated image signal is transmitted to the image-processing unit 21.

When the excitation light is emitted by the light-source unit 30, the excitation light component reflected by the subject is removed from the light incident on the exciting-light cut-off filter 53 by the exciting-light cut-off filter 53. In so doing, an optical image formed only by the autofluorescence component, autofluoresced by tissue to be observed, is captured by the imaging device 52.

As described above, the endoscope processor 20 can be connected to the normal endoscope (not depicted). The normal endoscope does not comprise the exciting-light cut-off filter 53, as compared with the autofluorescence endoscope 50. Accordingly, when the normal endoscope is connected to the endoscope processor 20, an optical image formed by the reflected light of a subject illuminated by the first light component and/or the autofluorescence component autofluoresced by the subject illuminated by the excitation light is captured by the imaging device 52.

Next, the structure of the image-processing unit 21 is explained. The image-processing unit 21 comprises a first signal processing circuit 25 (the receiver), an image processing circuit 26, and a second signal processing circuit 27 (see FIG. 1).

The image signal transmitted from the imaging device is input to the first signal processing circuit 25. The first signal processing circuit 25 digitizes the received image signal. In addition, the first signal processing circuit 25 carries out predetermined data processing, such as A/D conversion processing, YC processing, and color interpolation processing, on the image data digitized from the image signal.

In addition, the first signal processing circuit 25 calculates an average luminance value of light received by the entire light-receiving surface on the basis of the received image signal. Then, the first signal processing circuit 25 generates a luminance signal corresponding to the calculated average luminance value, and transmits it to the light-amount control circuit 41. As described above, the light-amount control circuit 41 adjusts the aperture ratio of the diaphragm 33 on the basis of the received luminance signal. Furthermore, when a normal endoscope is connected to the endoscope processor 20, the light-amount control circuit 41 adjusts the duty of the laser source 32.

The image data having undergone predetermined data processing at the first signal processing circuit 25 are transmitted to the image processing circuit 26. The image processing circuit 26 has a flash memory (not depicted), which is used as a work memory for signal processing. The image data is stored in the flash memory.

The image processing circuit 26 carries out color separation processing on the image data stored in the flash memory. In the color separation processing, the image data is separated into red, green, and blue data components. After color separation processing, the image processing circuit 26 carries out predetermined data processing including white balance processing on the red, green, and blue data components separately. In white balance processing, the red and blue data components are separately multiplied by the gains determined in the initialization operation for white balance.

The image data having undergone predetermined data processing is transmitted to the second signal processing circuit 27. The second signal processing circuit 27 carries out predetermined data processing on the image data, such as clamp processing and blanking processing. In addition, the second signal processing circuit 27 converts the image data into an analog image signal. The image signal is transmitted to the monitor 11, on which an image corresponding to the image signal is displayed.

Next, the control of the light amount emitted by the light-source unit 30 using a normal endoscope is explained. As for an autofluorescence endoscope, the control is described later. As described above, the aperture ratio of the diaphragm 33 and the duty of the laser source 32 are adjusted according to the average luminance value when a white-light image should be observed.

In order to compare the average luminance value, a reference value is predetermined, and reference data corresponding to the reference value is stored in a ROM (not depicted) connected to the light-amount control circuit 41 and read by the light-amount control circuit 41 when a light amount must be controlled.

The light-amount control circuit 41 compares the average luminance value with the reference value. If the average luminance value is less than the reference value, the diaphragm motor 39 and the laser source 32 are driven so that the aperture ratio of the diaphragm 33 and the emitted excitation light amount rise. On the other hand, if the average luminance value is more than the reference value, the diaphragm motor 39 and the laser source 32 are driven so that the aperture ratio and the amount of the emitted excitation light diminish.

If the aperture ratio and the amount of the emitted excitation light are adjusted in unrelated fashion, the color temperature of the light emitted from the exit end of the light guide 51 will vary. In order to keep the color temperature constant, the ratio of the amounts of excitation light to the first light component shone from the exit end, hereinafter referred to as a first ratio (the second relation), should be kept constant.

Optical specifications of light guide 51 for a normal endoscope may differ greatly from that of an autofluorescence endoscope. Consequently, in order to keep the first ratio constant, the ratio of the amounts of excitation light to the first light components incident on the incident end, hereinafter referred to as the second ratio, should match the ratio determined according to the kind of endoscope connected to the endoscope processor 20 (the first relation).

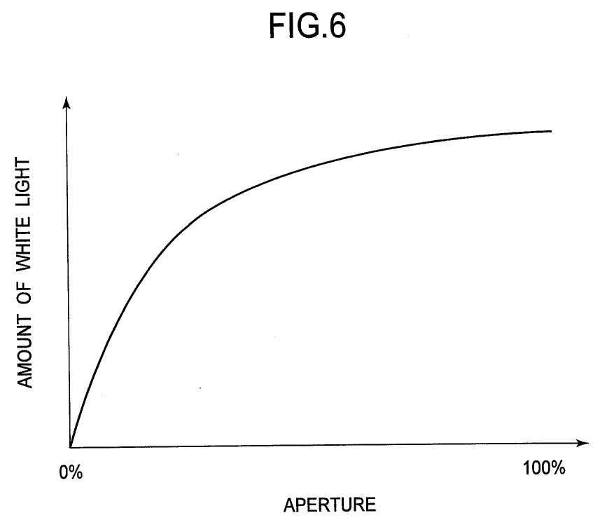

As described above, the amounts of the first light component and the excitation light are adjusted by changing the aperture ratio and the duty of the laser source 32. As shown in FIG. 6, the amount of the first light component varies nonlinearly with the aperture ratio. On the other hand, the amount of the excitation light varies linearly with the duty of the laser source 32.

Accordingly, in order to keep the first ratio constant, the aperture ratio and the duty should be adjusted so that the aperture ratio and the duty satisfy a specific correspondence. The specific correspondence is calculated by the initialization operation for white balance as described later. The light-source unit 30 comprises a first RAM (not depicted), and the specific correspondence is stored in the first RAM. When a white-light image is to be observed, the duty of the laser source 32 is determined according to the aperture ratio and the laser source 32 is driven at the determined duty.

When an autofluorescence endoscope is connected to the endoscope processor 20, only the aperture ratio is adjusted because the amount of light does not vary even if the duty is adjusted. As described above, the autofluorescence endoscope has the exciting-light cut-off filter 53, which removes the excitation light component. Consequently, because the excitation light components do not reach the imaging device 52, the amount of excitation light emitted by the laser source 32 does not have to be controlled.

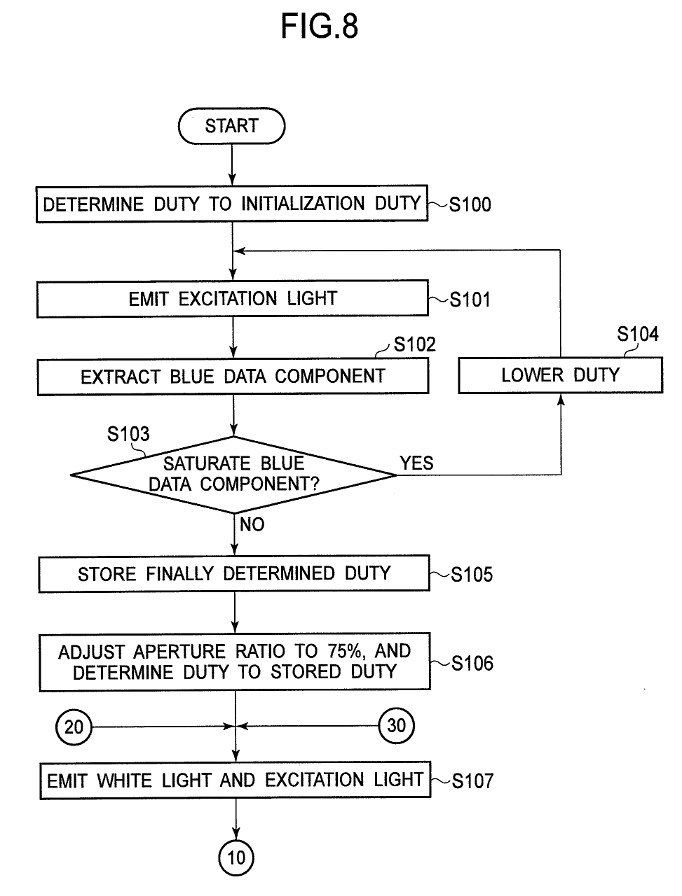

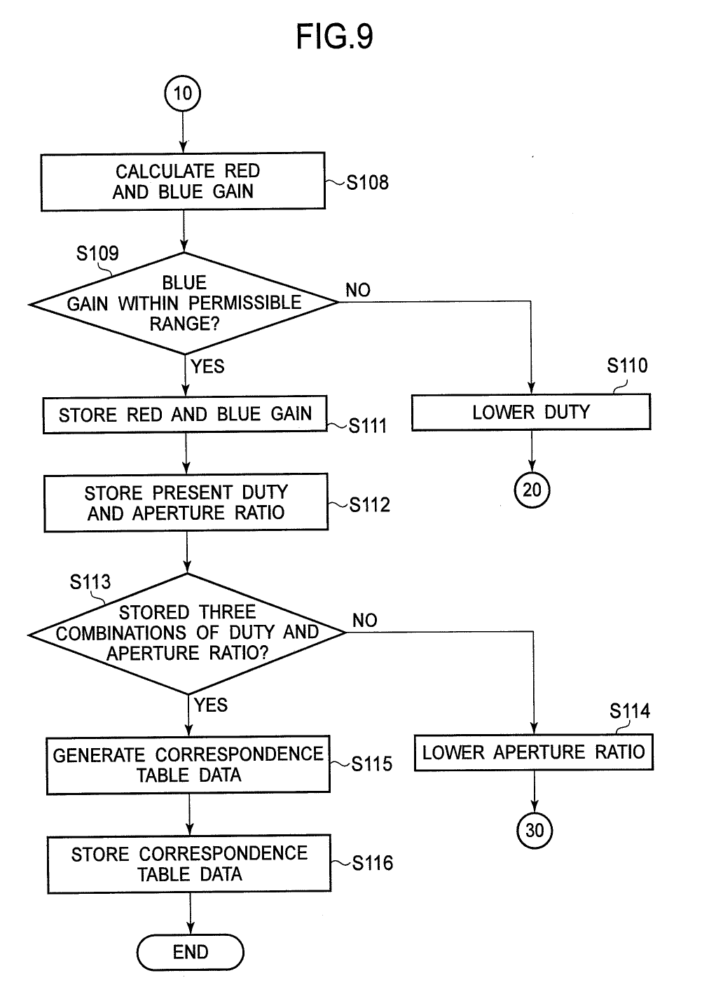

Next, the initialization operation for white balance carried out by the system controller 23 is explained using the flowcharts of FIGS. 8 and 9. In the initialization operation, the gains to multiply red and blue data components and the specific correspondence between the aperture ratio and the duty are determined.

The user is instructed to cover the head end of the insertion tube 56 with a white balance cover during the initialization operation. The white balance cover has a white interior. The initialization operation is carried out on the assumption that the head end is covered with the white balance cover. When a user inputs a command for ordering the initialization operation to the input block 24, the system controller 23 commences the initialization operation.

At step S100, the system controller 23 orders the light-amount control circuit 41 to determine a duty of the laser source 32 to the initialization duty predetermined on manufacturing.

At step S101 following step S100, the system controller 23 orders the laser source 32 via the light-amount control circuit 41 to emit the excitation light at the determined duty.

At step S102 following step S101, the system controller 23 orders the imaging device 52 via the imaging device driver 22 to capture an image of the inside of the white balance cover illuminated by the excitation light. In addition, the system controller 23 orders the image processing circuit 26 to extract the blue data components from the image signal. The blue data components are extracted, and the process proceeds to step S103.

At step S103, the system controller 23 determines whether or not the blue data components are saturated, in other words whether or not the blue data components have reached the maximum data level representable by the image processing circuit 26. If the blue data components are saturated, the process proceeds to step S104. At step S104, the system controller 23 orders the light-amount control circuit 41 to lower the duty of the laser source 32. After lowering the duty, the process returns to step S101. Since then, steps S101-S104 are repeated until the blue data components are not saturated.

If it is determined at step S103 that the blue data components are not saturated, the process proceeds to step S105. At step S105, the system controller orders the light-amount control circuit 41 to store the finally determined duty as a maximum adjustable duty in the first RAM (not depicted) connected to the light-amount control circuit 41.

At step S106 following step S105, the system controller 23 orders the light-amount control circuit 41 to drive the diaphragm motor 39 so that the aperture ratio is 75%. In addition, the system controller 23 orders the light-amount control circuit 41 to determine the duty to the maximum adjustable duty stored at step S105. After adjusting the aperture ratio and the duty, the process proceeds to step S107.

At step S107, the system controller 23 orders the lamp 31 via the power supply circuit 38 to emit white light and orders the laser source 32 via the light amount control circuit 41 to emit excitation light at the determined duty.

At step S108 following step S107, the system controller 23 orders the imaging device 52 via the imaging device driver 22 to capture an image of the interior of the white balance cover illuminated by the first light component and the excitation light. In addition, the system controller 23 calculates a red gain and a blue gain to multiply the red and blue data components on the basis of the captured image signal. After calculation of the gains, the process proceeds to step S109.

At step S109, the system controller 23 determines whether or not the blue gain is included in a permissible range predetermined on manufacturing. If the blue gain is out of the permissible range, the process proceeds to step S108. At step S108, the system controller 23 orders the light-amount control circuit 41 to lower the currently determined duty of the laser source 32. After lowering the duty, the process returns to step S107. After that, steps S107-S108 are repeated until the blue gain is included in the permissible range.

Only the blue gain is compared with the permissible range because the laser source 32 alone shines the blue light component on a subject, as explained next. If the blue light component is supplied by the lamp 31, the calculated blue gain will be adequate. However, in the endoscope processor 20, the amount of the blue light component in the white light shone on the subject may differ greatly from those of the red and green light components in the white light. Consequently, the calculated blue gain may be quite different than the blue gain calculated with the white light supplied by only the lamp 31 on the subject. If the white balance processing is carried out using blue gain that is far off, more blue color noise may appear in the generated image. Consequently, the range of blue gain necessary for avoiding blue color noise in the generated image is predetermined as the permissible range.

If it is determined at step S109 that the blue gain is within the permissible range, the process proceeds to step S111. At step S111, the system controller 23 orders a second RAM (not depicted) connected to the image processing circuit 26 to store the red and blue gains calculated at step S108. After storage, the process proceeds to step S112.

At step S112, the system controller 23 orders the first RAM to store the present duty and the present aperture ratio which correspond to each other. After storage, the process proceeds to step S113.

At step S113, the system controller 23 determines whether or not the three combinations of corresponding duty and aperture ratio have been stored in the first RAM.

If the three combinations have not been stored, the process proceeds to step S114. At step S114, the system controller 23 orders the light-amount control circuit 41 to drive the diaphragm motor 39 so that the aperture ratio is lowered by 25%. Consequently, if the present aperture ratio is 75% and 50%, the aperture ratio is adjusted to 50% and 25%, respectively. After adjusting the aperture ratio, the process returns to step S107. After that, steps S107 to S114 are repeated until the three combinations have been stored.

On the other hand, if it is determined at step S113 that the three combinations have been stored, the process proceeds to step S115. At step S115, the system controller 23 generates correspondence table data corresponding to the specific correspondence between the aperture ratio and duty on the basis of the three different duties corresponding to the aperture ratios of 75, 50, and 25%. After generating the correspondence table data, the process proceeds to step S116.

At step S116, the system controller 23 stores the correspondence table data in the first RAM. When the correspondence table data is stored, the initialization operation for white balance ends.

The above initialization operation is carried out when a normal endoscope is connected to the endoscope processor 20. When an autofluorescence endoscope is connected to the endoscope processor 20, an adjustment of the excitation light emitted by the laser source 32 is unnecessary because the exciting-light cut-off filter 53 is mounted. Accordingly, when an autofluorescence endoscope is connected to the endoscope processor 20, the above initialization operation is carried out with the omission of steps S102, S103, S104, S109, S110, S112, S113, S114, S115, and S116.

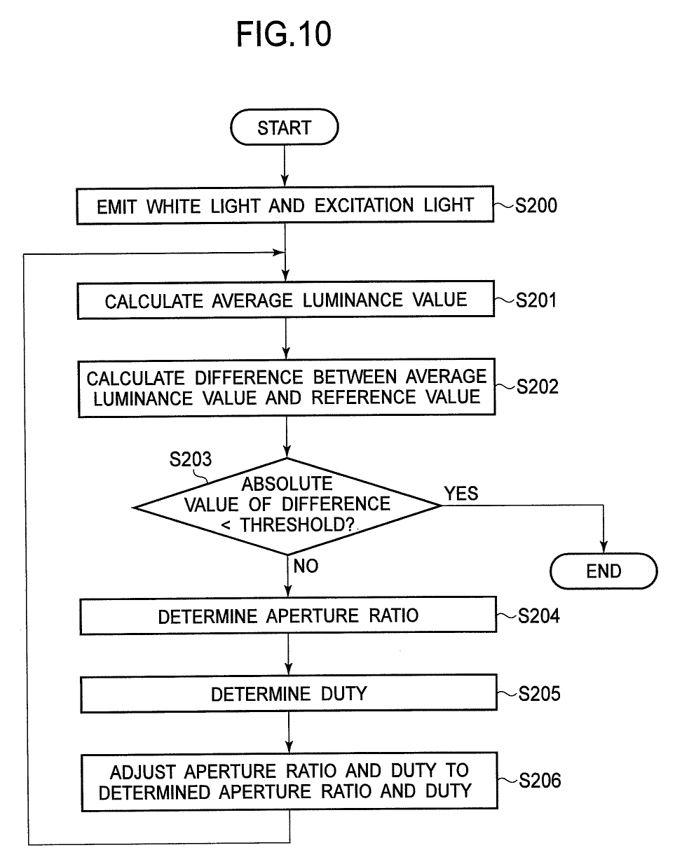

Next, the operation for control of light amount, which is carried out by the system controller 23 when a captured image is displayed using a normal endoscope, is explained using the flowcharts of FIG. 10.

At step S200, the system controller 23 orders the lamp 31 and the laser source 32 via the light-amount control circuit 41 to emit the white light and the excitation light, respectively.

At step S201 following step S200, the system controller 23 orders the imaging device 52 via the imaging device driver 22 to capture a subject illuminated by the first light component and the excitation light for generating an image signal. In addition, the system controller 23 orders the first signal processing circuit to calculate the average luminance value on the basis of the generated image signal. After calculation of the average luminance value, the process proceeds to step S202.

At step S202, the system controller 23 orders the light-amount control circuit 41 to calculate the difference between the average luminance value and the reference value.

At step S203 following step S202, the system controller 23 orders the light-amount control circuit 41 to determine whether or not the absolute value of the calculated difference is less than a threshold. If the difference is less than the threshold, the operation for control of light amount ends. On the other hand, if the difference is greater than or equal to the threshold, the process proceeds to step S204.

At step S204, the system controller 23 orders the light-amount control circuit 41 to determine the aperture ratio of the diaphragm 33 according to the difference calculated at step S202. After the determination, the process proceeds to step S205.

At step S205, the system controller 23 orders the light-amount control circuit 41 to determine the duty of the laser source corresponding to the aperture ratio determined at step S204 on the basis of the correspondence table data generated in the initialization operation for white balance.

At step S206 following step S205, the system controller 23 orders the light-amount control circuit 41 to drive the diaphragm motor 39 so that the aperture ratio of the diaphragm matches the aperture ratio determined at step S204 and to drive the laser source 32 at the duty determined at step S205.

In the above embodiment, an acceptable white image can be produced by connecting a normal endoscope to the endoscope processor by adjusting the duty of the laser source 32 according to the aperture ratio of the diaphragm.

In addition, in the above embodiment, a mechanism for moving the dichroic mirror 35 is unnecessary because the dichroic mirror 35 can be fixed. Accordingly, faults of light-source unit 30 can be reduced, the latency to switch light sources can be shortened, the number of parts for the light-source unit 30 can be reduced, and the manufacturing cost is reduced.

In addition, in the above embodiment, blue color noise can be reduced because the amounts of the first light component and the excitation light emitted by the light-source unit 30 can be separately adjustable. In general, the sensitivity of an imaging device for the blue light component may be relatively lower than for green and red. So, when a subject illuminated by white light consisting of practically the same amounts of blue, green, and red light components is imaged, relatively large blue gain is used. Accordingly, blue color noise would increase and become noticeable. On the other hand, in the above embodiment, the blue color noise will be reduced by relatively increasing the amount of excitation light emitted by the light-source unit 30.

The specific correspondence between the aperture ratio of the diaphragm 33 and the duty of the laser source 32 is determined by the system controller 23 in the initialization operation for white balance in the above embodiment. However, the specific correspondence can be determined by other methods. The same effect can be achieved as long as the duty is adjusted according to the aperture ratio so that the adjusted duty and the current aperture ratio satisfy the specific correspondence. For example, the specific correspondence for each electronic endoscope can be determined on manufacturing and stored in an endoscope memory mounted in the electronic endoscope. When the endoscope is connected to the endoscope processor 20, the light-amount control circuit 41 reads the specific correspondence and uses it for adjusting the duty.

The amounts of the first light component and the excitation light shone on a subject are adjusted by changing the aperture ratio of the diaphragm 33 and the duty of the laser source 32, respectively, in the above embodiment. However, the amounts can be adjusted using any other devices for light control. As long as the amounts are controlled so that the second ratio matches a ratio determined for each endoscope, the same effect can be achieved.

The white light and the excitation light are simultaneously emitted by the light-source unit 30 and the amounts of color components of the received light are simultaneously detected on the initialization operation for white light in order to determine the specific correspondence, in the above embodiment. However, the white light and the excitation light can be separately emitted and the amounts of color components of the received light can be separately detected.

The duty of the laser source 32 is adjusted according to the aperture ratio of the diaphragm 33 for observing a white-color image in the above embodiment. However, even if the aperture ratio is adjusted according to the duty, the effect of the above embodiment can be achieved. However, the adjustment of the duty based on the aperture ratio is achieved more quickly than based on the duty.

The number of combinations of aperture ratio and duty actually detected on the initialization operation for white balance is three in the above embodiment. However, the number is not limited to three. In the initialization operation, by determining the adequate duties for a certain blue gain for three more different aperture ratios and by using the combinations of the aperture ratio and the duty for determining the specific correspondence, the amount of the excitation light shone on a subject is more adequately controlled.

The duty of the laser source 32 is adjusted when the aperture ratio is set to 75, 50, and 25% in the initialization operation for white balance in the above embodiment. However, the aperture ratio to be set is not limited to 75, 50, and 25%. The duty may be adjusted if the amount of the first light incident on the light guide 51 is kept constant and the inside of the white balance cover is captured.

The light-source unit 30 can simultaneously or separately emit the first light component and the blue excitation light in the above embodiment. However, the light-source unit 30 may emit at least two different kinds of light which include at least one of red, green, and blue light components.

Although the embodiment of the present invention has been described herein with reference to the accompanying drawings, obviously many modifications and changes may be made by those skilled in this art without departing from the scope of the invention.

The present disclosure relates to subject matter contained in Japanese Patent Application No. 2008-050210 (filed on Feb. 29, 2008), which is expressly incorporated herein, by reference, in its entirety.

1. An endoscope light source system, comprising:
first and second light sources that make first and second lights incident on an incident end of a light guide mounted in an endoscope, respectively, the first and second lights having first and second wavelength bands, respectively; and
an adjustment circuit that adjusts the incident amounts of the first and/or second lights to satisfy a first relation if the first and second lights are simultaneously made on the incident end, the incident amounts of the first and second lights being the amounts of the first and second lights made incident on the incident end.

2. An endoscope light source system according to claim 1, wherein the first relation is determined so that emission amounts of the first and second lights satisfy a second relation upon making the first and second lights simultaneously incident on the incident end, the emission amounts of the first and second lights being the amounts of the first and second lights emitted from an exit end of the light guide, respectively.

3. An endoscope light source system according to claim 2, wherein a ratio of the emission amount of the first light to the emission amount of the second light is constant in the second relation.

4. An endoscope light source system according to claim 1, further comprising a determination circuit that determines the first relation so that a gain is included in a predetermined range, said gain being multiplied by reflected light components of the second light in an optical image for white balance processing, said white balance processing being carried out for the optical image of a subject illuminated by the first and second light emitted from the exit end upon making the first and second lights incident on the incident end.

5. An endoscope light source system according to claim 4, further comprising a switch which a command for ordering the determination circuit to determine the first relation is input to.

6. An endoscope light source system according to claim 1, further comprising a determination circuit that determines the first relation on the basis of a first and second light received amounts, the first and second light received amounts being an amount of first and second subject light received from a subject upon making the first and second lights incident on the incident end and illuminating the subject with the first and second lights, respectively.

7. An endoscope light source system according to claim 6, further comprising a detector that receives the first and second subject light and detects the first and second light received amounts.

8. An endoscope light source system according to claim 6, further comprising a receiver that receives first and second amount signals from a detector, the first and second amount signals corresponding to the first and second light received amounts, the detector receiving the first and second subject lights and detecting the first and second light amounts.

9. An endoscope light source system according to claim 1, wherein the adjustment circuit adjusts the incident amount of the first light of a third light received amount, the third light received amount being the amount of third subject light received from a subject upon making the first and second lights simultaneously incident on the incident end and illuminating the subject with the first and second lights simultaneously.

10. An endoscope light source system according to claim 1, wherein the incident amount is adjusted by changing a size of an aperture of a diaphragm.

11. An endoscope light source system according to claim 1, wherein the incident amount is adjusted by controlling an amount of the first and/or second light emitted by the first and/or second light sources.

12. An endoscope light source system according to claim 1, wherein the first and second lights include at least one of red, green, and blue light components.

13. An endoscope unit, comprising:
an endoscope that comprises a light guide;
first and second light sources that make first and second lights incident on an incident end of the light guide, respectively, the first and second lights having first and second wavelength bands, respectively; and
an adjustment circuit that adjusts the incident amounts of the first and/or second lights so that the emission amounts of the first and second lights satisfy a second relation upon making the first and second lights simultaneously incident on the incident end, the incident amounts of the first and second lights being the amounts of the first and second lights made incident on the incident end, the emission amounts of the first and second lights being the amounts of the first and second lights emitted from an exit end of the light guide, respectively.
